(19) United States
(12) Patent Application Publication
(10) Pub. No.: US 2006/0258969 A1
(43) Pub. Date: Nov. 16, 2006

(54) Shower patch

(75) Inventors: Ivan E. Brown, Spirit Lake, IA (US); Amy Wheaton, Spirit Lake, IA (US); Terry Kounkel, Spirit Lake, IA (US); Matt Henry, Arnolds Park, IA (US); Becki Huisenga, Spirit Lake, IA (US)

Correspondence Address: MCKEE, VOORHEES & SEASE, P.L.C., 801 GRAND AVENUE, SUITE 3200, DES MOINES, IA, 50309-2721, US

(73) Assignee: BROWN MEDICAL INDUSTRIES, Spirit Lake, IA (US)

(21) Appl. No.: 11/127,923

(22) Filed: May 12, 2005

(51) Int. Cl. A61F 15/00; A61F 13/00

(52) U.S. Cl. 602/57; 602/54

(57) ABSTRACT

A moldable body patch to provide moisture protection for wound sites. It uses a non-linear shaped preferably medical grade flexible film piece having upper, preferably middle and lower portions and a pull tab permanently adhered to the upper portion of the film piece. A backing paper is removably adhered to the middle portion and to the lower portion which may be removed to reveal an adhesive that will provide skin adherence in a sealable relationship to prevent underlying wounds from moisture contact. When finished, the film piece may be conveniently removed by pulling on the pull tab permanently adhered to the upper portion of the film piece.

### FIELD OF THE INVENTION

This invention relates to moldable body patches to provide moisture protection for wound sites during for example showering.

### BACKGROUND OF THE INVENTION

Shower patches have been used in the past to cover wound sites but those have worked less than satisfactory. For example, typical prior art shower patches often involve square film pieces using 90° angle tear-away strips to reveal underlying adhesive that covers just the strip area, leaving a blank spot in each corner. The problem with such shower patches are two-fold. First, sharp angles on the patches seem to provide an increased propensity for moisture leakage due to the blank spot. Second, square angles also cause improper skin adherence.

Accordingly, it is a primary object of the present invention to provide an improved shower patch which may be easily applied, easily removed, and one which will not interfere with the underlying wound site during use.

Another objective of the present invention is to provide an improved shower patch for wound sites which has greater ease of application, which better fits the contour of the human anatomy, and which provides less propensity for leakage which allows moisture access to the underlying wound.

A method and means of accomplishing each of these objectives as well as others will become apparent from the detailed description of the invention which follows.

### SUMMARY OF THE INVENTION

A preferred embodiment of the invention involves a moldable body patch to provide moisture protection for wound sites. The patch has a non-linear shaped medical grade film piece, preferably elliptical shaped, which has upper, lower and middle portions. Middle and lower portions have a backing paper removably adhered to the film piece while the upper portion has a pull tab permanently adhered to the upper portion. In use, the user removes the backing from the middle backing portion, removes the lower backing portion paper and applies the patch over the wound site with the tab appearing above the wound dressing. The elliptical shape fits the human anatomy, and since there is no adhesive directly underneath the wound site, there is no interference with the wound dressing. When finished the pull tab is pulled directly downward and the shower patch removed.

### BRIEF DESCRIPTION OF THE DRAWINGS

FIG. 1 is a plan view of the shower patch.

FIG. 2 is a sectional view of the patch along line 2-2 of FIG. 1.

FIG. 3 is a view of the shower patch applied over a wound dressing as illustrated in the general lower abdomen area.

FIG. 4 shows how the permanently adhered pull on tab at the top may be conveniently used to pull off the shower patch for discard when use is completed.

### DETAILED DESCRIPTION OF THE PREFERRED EMBODIMENT

For purposes of promoting and understanding of the principles of the invention reference will be made to the embodiment illustrated in the drawings and specific language will be used to describe the same. It will nevertheless be understood that no limitation of the scope of the invention is thereby intended, such alterations and further modifications in the illustrated embodiment, and such further applications of the principles of the invention are contemplated as being within the scope of the invention, as are modification that would normally occur to one skilled in the art to which the invention relates. The limits of the invention are defined by the claims, not the description of the preferred embodiment.

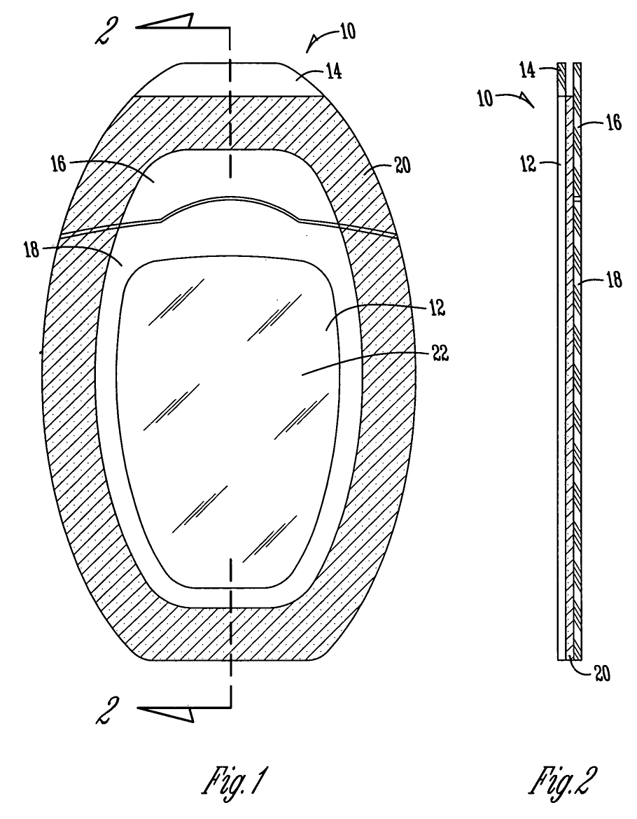

Referring now more particularly to FIG. 1 there is shown a shower patch 10 which is comprised of a non-linear shaped medical grade thin flexible film piece 12. It is preferred that the thin flexible film piece 12 be non-rectangular which is the reason for the descriptor “non-linear”; however, such is intended to include a variety of non-linear shapes including circular, oval, elliptical, etc. The film piece 12 has a permanently adhered separate end distinct pull tab 14 and in the preferred embodiment shown in FIG. 1, other distinct backing paper portions, i.e., a middle backing paper 16 and a lower backing paper 18. Preferably the film piece 12 is transparent. Located between film piece 12 and middle backing paper 16 and lower backing paper 18 is a medical grade adhesive 20. In fact both the film piece 12 and the adhesive 20 are medical grade. The adhesive is one which removably attaches over backing paper 18 and middle backing paper 16 in the sense that they can be peeled away leaving adhesive on the film piece 12, of sufficient stickiness then it can be successfully applied to the human anatomy to provide sealable adhererice to body skin.

The adhesive 20 is applied to coincide with the outer surface area of the middle backing paper 16 and the lower backing paper 18. Thus the window or central portion 22 of film piece 12 does not have adhesive on its backside.

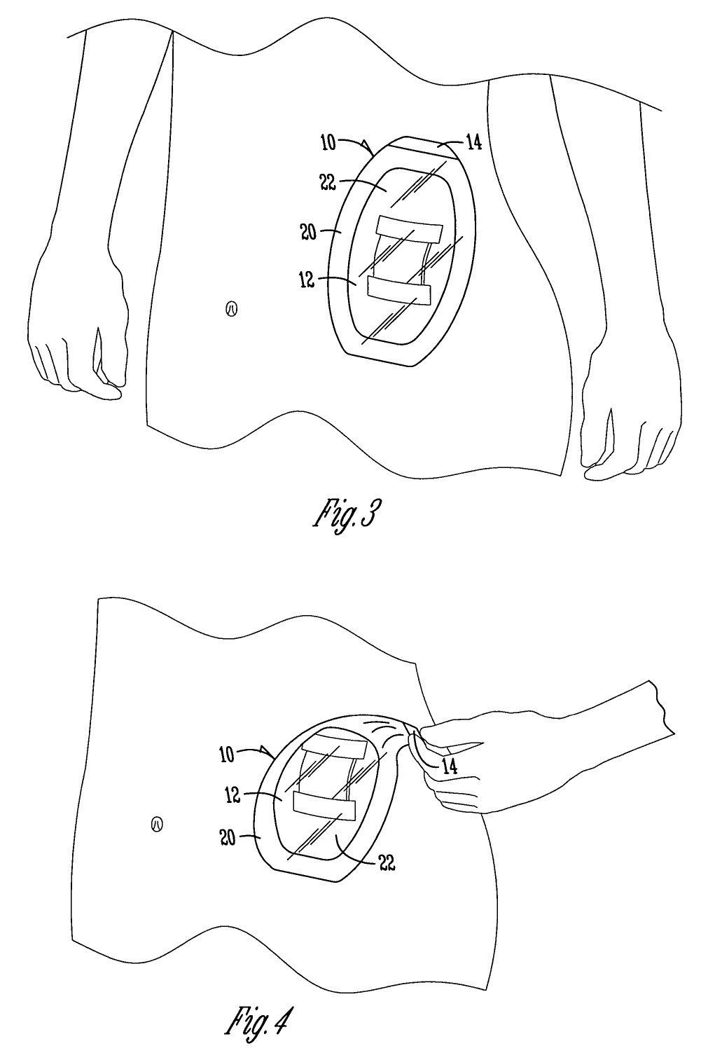

FIG. 3 shows the shower patch with middle backing paper 16 and lower backing paper 18 removed and applied to a lower abdomen wound for coverage so that the patient/user may shower and have wound dressing protection. It can be seen in FIG. 3 that the generally non-linear elliptical shape provides a configuration that easily fits the human anatomy without sharp corner edges which, based upon experience, have a propensity to form leakage areas.

FIG. 4 shows the convenience of the pull tab 14 so that it may be simply grabbed and pulled downward to remove the shower patch which can then be discarded.

The patch is conveniently packaged, and in some instances, five per package is typical. Typical dimensions are a coverage area of 5 inches by 8 inches or 3½ inches by 6 inches. It is best used when applied to clean, dry and shaved skin. Its shape allows it to conform to body contours and not stretch over the covered area. As a result, leakage is minimized.

Medical grade film which is used is hypoallergenic and biocompatible. It is thin and flexible to prevent tunneling in stressed areas and is preferably an ethyl vinyl acetate (EVA) film. The medical grade adhesive which is used is applied through a lamination process and has been through cytotoxicity and skin sensitization testing. It is biocompatible with the skin and not overly aggressive reducing the possibility of irritation. The inner paper backing which is used is set out in two pieces for easy removal. It can therefore be seen that the invention accomplishes at least all of its stated objectives.

1. A moldable body patch to provide moisture protection for wound sites, comprising:
a non-linear shaped film piece having a perimeter and upper, lower and middle portions;
a pull tab permanently adhered substantially within the perimeter of the non-linear shaped film piece to the upper portion of said film piece;
a middle backing paper removably adhered to said film piece;
a lower backing paper removably adhered to said film piece;
said middle and lower backing papers being removably adhered by an adhesive which is revealed upon removal of said papers to allow removable but sealable adherence to body skin.

2. The body patch of claim 1 wherein the film piece is transparent.

3. The body piece of claim 1 wherein the non-linear shaped film piece is generally elliptical in shape.

4. The body patch of claim 1 wherein the lower backing paper is a perimeter strip with the center cut out to reveal the film piece.

5. The body patch of claim 4 wherein the adhesive of said lower back paper is only under the perimeter strip and not under the central portion.

6. The body patch of claim 1 wherein the film piece is biocompatible.

7. The body patch of claim 1 wherein the adhesive is biocompatible.

8. (canceled)

9. A moldable body patch to provide moisture protection for wound sites comprising:
a non-linear shape medical grade film piece having a perimeter and upper and lower portions;
a pull tab permanently adhered substantially within the perimeter of the non-linear shape medical grade film piece to the upper portion of said film piece;
a backing paper removable adhered to the lower portion of said film piece by a medical grade adhesive which is revealed upon removal of the backing paper to allow removable and sealable adherence to body skin.

10. A method of providing moisture protection for wounds during showering, said method comprising:
providing a moldable body patch that is a non-linear shaped medical grade film piece having a perimeter and upper and lower portions, a pull tab permanently adhered substantially within the perimeter of the non-linear shaped medical grade film piece to the upper portion of said film piece, and a lower backing paper removably adhered to said film piece;
removing the lower backing paper to reveal a perimeter adhesive and placing the non-linear film piece with the remaining permanently adhered pull tab over a wound site to provide a removable but sealable adherence to body skin surrounding the wound site; and thereafter
pulling on the permanently adhered pull tab to remove the moldable body patch when no longer needed.

11. The body patch of claim 1 wherein the adhesive is a medical grade adhesive.

12. The body patch of claim 1 wherein the film piece is a medical grade adhesive.
